(19) United States
(12) Patent Application Publication
(10) Pub. No.: US 2002/0032915 A1
(43) Pub. Date: Mar. 14, 2002

(54) Method of screening for large offspring syndrome

(76) Inventors: Thomas Gerard McEvoy, Aberdeen (GB); Kevin Donald Sinclair, Aberdeen (GB); Lorraine Elizabeth Young, Innerleithen (GB); Ian Wilmut, Edinburgh (GB)

Correspondence Address: HALE AND DORR, LLP, 60 STATE STREET, BOSTON, MA, 02109

(21) Appl. No.: 09/816,971

(22) Filed: Mar. 23, 2001

Related U.S. Application Data
(63) Continuation of application No. PCT/GB99/03185, filed on Sep. 23, 1999.

(30) Foreign Application Priority Data
Sep. 25, 1998 (GB) 9820988.5

(51) Int. Cl. A01K 67/027; C12Q 1/68; A61K 48/00

(52) U.S. Cl. 800/21; 800/14; 800/15; 514/44; 435/6

(57) ABSTRACT

A method is provided for the diagnosis of Large Offspring Syndrome (LOS) in animals prepared by nuclear transfer or in animals prepared by methods of in vitro fertilization or culture which comprises analyzing Insulin-like growth factor-2 receptor (Igf2r) gene expression in the animal embryo or in a biological sample from the embryo.

The present invention relates to a method of screening for abnormal development in an animal embryo or embryo-derived pluripotent cell-line and to methods for manipulating animal embryos or cells in in vitro culture which incorporate the screening method. The invention also relates to uses of the screening method in testing whether cell-culture environments or manipulation protocols induce abnormal development in an animal embryo.

Bovine and ovine embryos exposed to a variety of unusual environments prior to the blastocyst stage have resulted in the development of abnormally large offspring (Young et al Reviews of Reproduction—in press (1998)) which can also exhibit a number of organ defects, including skeletal and facial malformations (Walker et al Theriogenology 45 111-120 (1996)), over-muscling and alterations in muscle fibre compositions (Maxfield et al Am. J. Physiol. 274 E 1 121-E 1 123 (1998)), cerebellar hypoplasia (Schmidt et al Theriogenology 46 527-539 (1996)), liver and cardiovascular defects (van Soom et al Theriogenology 41 855-867 (1994); Wilmut et al Nature 385 810-813 (1997); Campbell et al Nature 380 383 (1996); Cibelli et al Science 280 1256-1258 (1998)), urogenital tract defects (Campbell et al Nature 380 383 (1996)), pulmonary defects (Cibelli et al Science 280 1256-1258 (1998)), and placental defects including polyhydramnios, hydroallantois and enlarged placentomes (Cibelli et al Science 280 12561258 (1998)). Enlargement of several organs has been reported (Farin, P. W. and Farin, C. E., Biology of Reproduction 52 676-682 (1995); Cibelli et al Science 280 1256-1258 (1998)) and associated features include lengthened gestation, dystocia (Kruip, T. A. M. and den Haas, J. H. G., Theriogenology 47 43-52 (1997)) and physiological defects especially relating to thermoregulation, energy balance, respiration and metabolic pH regulation (Garry et al Theriogenology 45 141-152 (1996)). Perinatal mortality is a particular problem and prenatal losses are high (Walker et al Theriogenology 45 111-120 (1996)). This group of symptoms is now generally described as Large Offspring Syndrome (LOS), although it has sometimes been referred to as Large Calf Syndrome (Willadsen et al Theriogenology 35 161-170 (1991)). Not all features are observed in each case of LOS and in some cases, features are observed in the absence of obviously large size.

Manipulation of the pre-implantation embryo in nuclear transfer, in vitro culture of embryos, asynchronous embryo transfer or progesterone treatment of the mother soon after ovulation have all now been associated with such problems (reviewed by Walker et al Theriogenology 45 111-120 (1996)), as has the administration of high urea diets to the mother (McEvoy et al Animal Reproduction Science 47 71-90 (1997)). While alterations to the embryo environment in vivo have been of interest experimentally, the problems associated with LOS are of particular commercial importance in relation to in vitro treatments of the pre-implantation embryo, both in nuclear transfer and in vitro production. In vitro production can include any or all of the following three stages: (1) in vitro maturation (IVM) of the oocyte; (2) in vitro fertilisation (IVF) of the oocyte; and/or (3) in vitro culture (IVC) of the fertilised oocyte, generally to the blastocyst stage. Nuclear transfer can also include periods of in vitro culture, either of the oocyte prior to, during and subsequent to manipulation procedures or of the reconstructed embryo. Culture of the nuclear transfer-reconstructed embryo can either be in vitro or in vivo in a reproductive tract, usually ligated in a mammalian (usually sheep or rabbit) oviduct. Both provide unusual embryo environments and have been associated with the symptoms of LOS.

Fetal growth syndromes have also been described in humans and mice. However these are not associated with pre-implantation embryo manipulation but are, respectively, the result of either chromosomal and other genetic abnormalities or experimental genetic manipulation. The absence of fetal overgrowth in humans and mice following embryo manipulation or culture may be due to intrinsic species differences from cattle and sheep, or, alternatively, to differences in the protocols used. For example, livestock embryos are usually transferred to recipients as blastocysts, whereas human embryos have historically been returned to the mother at around the four-cell stage and so it is possible that the human embryo is not exposed to the perturbing factor(s) at a critical period. Mouse embryos are cultured routinely to the blastocyst stage but all commonly-used media are serum free. Most cases of cattle and sheep LOS associated with in vitro embryo culture have involved exposure to co-cultured cells and/or serum (for example, see Walker et al Theriogenology 45 111-120 (1996)), which presumably produce and/or contain the perturbing factors. Thus, it is possible that the same factors affecting bovine and ovine embryos could also affect embryos from other species in a similar manner if the culture conditions are significantly altered.

Perturbed fetal growth has important clinical significance in terms of the delivery and survival of offspring. Commercially, it is of considerable relevance in the production of bovine and ovine embryos by in vitro production (IVP), including in vitro maturation (IVM) or in vitro fertilisation (IVF). The technique may become increasingly relevant in the expanding use of this technology in other species, for example, in valuable individuals such as in horses (Hinrichs Theriogenology 49 13-21 (1998)) and also in endangered species such as the panda (Saegusa Nature 394 409 (1998)). LOS is also likely to assume greater importance in the production of transgenic animals using nuclear transfer technology. The phenomenon of LOS may well be relevant to human IVF given that the events of the pre-implantation period can affect the future prospects for offspring. For example, conventional human IVF can result in the production of babies with relatively low birth weight.

As this technology becomes increasingly invasive (e.g. intracytoplasmic sperm injection, cytoplasmic reduction, in vitro culture to the blastocyst stage, embryo biopsy, pre-implantation diagnosis), the health of the offspring is increasingly at risk (Walker et al Theriogenology 45 111-120 (1996); Handyside et al Trends in Genetics 13 270-275 (1997)).

Embryo-derived embryonal stem (ES) cells and embryonal germ (EG) cells in culture can also be used to produce transgenic or non-transgenic mice or other animals (Nagy et al Proc. Nat'l. Acad. Sci. USA 90 8424-8428 (1993), Dean et al Development 125 2273-2282 (1998)) or cells and tissues from such organisms. Embryonic stem (ES) cells are pluripotential cell lines isolated from the inner cell mass cells of blastocyst-stage embryos (Evans, M. J. and Kaufman, M. H., Nature 292 154-156 (1981)), morulae (Eistetter, H. R. Dev. Growth Differ. 31 275-282 (1989)) or isolated epiblast cells (Brook, F. A. and Gardner, R. L. Proc. Nat'l Acad. Sci. USA 94 5709-5712 (1997)). The establishment of these lines and lines of pluripotential embryonal germ (EG) cells, derived from mouse primordial germ cells has enabled genetic modification of these cell lines in culture and the production of transgenic mice in which specific genes have been altered, introduced or replaced (McWhir, J., Roslin Institute Annual Report 1995-1996 80-83 (1996); L. M. Houdebine (ed) Transgenic Animals: Generation and Use, Overseas publishers Association, Amsterdam (1997)). While so far only ES and EG cells from mice have been able to contribute to the germ-line and thus produce a route to transgenic animals, there is currently much interest in the production of pluripotent cell lines from the inner cell mass or primordial germ cells of other species. Recently, there have been reports of chimeric pigs (Golueke et al Theriogenology 49 238 (1998)) and cattle (Cibelli et al Nature Biotechnology 16 642-646 (1998)) being produced using ES cell-like cells, derived from blastocyst inner cell mass cells. Since both in vitro culture (in the presence of both serum and co-cultured cells) and nuclear transfer techniques were used in the production of the cattle ES cell-like cells, there is a high risk that such techniques may result in the abnormalities characteristic of Large Offspring syndrome. This also applies to any future isolation of EG cells from primordial germ cells, also embryo derived. In mice, changes in ES cell-lines during the derivation or culture phases may be sufficient to induce abnormalities with some similarities to LOS (Nagy et al Proc. Nat'l. Acad. Sci. USA 90 8424-8428 (1993), Dean et al Development 125 2273-2282 (1998)), without in vitro culture of the resulting embryo (since the embryo culture techniques used in these and other mice studies have not been reported to induce a phenotype similar to LOS). Since it has recently been suggested that genetically-modified bovine ES cells may provide a therapeutic source of tissue for xenotransplantation to treat human disease (Cibelli et al Nature Biotechnology 16 642-646 (1998)), screening methods to ensure normal development may be of considerable value Pluripotent human EG cells and ES cells are being actively sought and it has been suggested that these could provide an “unlimited source of in-vitro-derived differentiated cells to treat specific diseases by transplantation” (First, N. L. and Thomson, J., Nature Biotechnology 16 620-621 (1998)). Furthermore, the possibility that a human or other oocyte may be able to reprogramme a nucleus from a differentiated human cell in a similar manner to that demonstrated recently in cattle (Cibelli et al Science 280 1256-1258), sheep (Campbell et al Nature 380 64-66 (1996); Wilmut et al Nature 385 810-813 (1997); and Schnieke et al Science 278 2130-2133 (1997)) and mice (Wakayama et al Nature 394 369-373 (1998)), has also been suggested as having important therapeutic applications (First, N. L. and Thomson, J., Nature Biotechnology 16 620-621 (1998)).

Other types of stem cells have been suggested which may be used to generate fetuses, cells or tissues, which may or may not be transgenic. These include embryonic and fetal-derived stem cells as well as those which persist in the adult animal. Examples include gonadal stem cells, haematopoietic stem cells, muscle stem cells, epidermal stem cells and neuronal stem cells (WO 94/24274). Since use of these stem cells in production of fetuses, cells or tissues, by methods such as embryo aggregation, blastocyst injection or nuclear transfer, all involve periods of in vitro embryo and/or cell culture, these cells may also potentially be perturbed to induce phenotypes similar to LOS.

Fetal overgrowth in humans and mice has resulted from alterations in the expression of several genes. The fetal overgrowth observed in Beckwith-Weidmann syndrome has been attributed to the H19 and/or Igf2 genes (Reik et al Human Molecular Genetics 4 2379-2385 (1995); Eggenschwiler et al Genes and Development 11 3128-3142 (1997); Sun et al Nature 389 809-815 (1997)) and to the GPC3 gene in Simpson-Golabi-Behmel syndrome (Pilia et al Nature Genetics 12 241-247 (1996)). In addition, mice that are null mutants for H19, Igf2 and Igf2r all have marked growth phenotypes. For example, H19 null mutants are 27% heavier (Leighton et al Nature 375 34-39 (1995)) and Igf2 null mutants are 40% lighter (De Chiara et al Cell 64 849-859 (1991)) than their respective wild type counterparts. Experimental deletion of the maternal Igf2r allele in mice also leads to a 30% increase in mean birthweight and elevated plasma concentration of the fetal mitogen, insulin-like growth factor II (IGF-II) (Lau et al Genes and Development 8 2953-2963 (1994). With the exception of GPC3, all of these genes have been shown to be imprinted, at least in the mouse. Parental (genomic) imprinting is the process by which the differential expression of maternal and paternal alleles occurs at certain genetic loci in mammalian embryos and cells (Moore, T. and Reik, W. Rev. Reprod. 1 73-77 (1996)). Aberrant expression of imprinted genes has been implicated in a range of embryonic and fetal abnormalities. Although the proximate functions of the products encoded by a significant proportion of imprinted genes are largely unknown, at least seven imprinted genes identified to date are thought to affect growth. Igf2, Mas and Ins2 are all paternally expressed and enhance growth rates. By analogy, Ins1 is also expected to be a growth enhancer. Igf2r, H19, p57KIP2 and Mash2 are all expressed from the maternally-derived genome and reduce growth rates. (Hurst et al Nature Genetics 12 234-237 (1996). In addition, several DNA binding protein genes which undergo imprinting, such as WT1, ZNF127 and Mash2, have been identified as having potential effects at other genomic loci suggesting a further layer of regulation (Moore, T. and Reik, W. Rev. Reprod. 1 73-77 (1996)) and therefore of potential deregulation. One of the components of the imprinting process is the methylation of DNA at CpG dinucleotides (Moore, T. and Reik, W. Rev. Reprod. 1 73-77 (1996)). In addition to their known effects on fetal growth, the imprinted genes (or those identified as imprinted in other species) have been suggested as candidates for involvement in the Large Offspring, syndrome in cattle and sheep as many imprinted genes undergo significant, allele-specific changes in their DNA methylation during early embryogenesis when imprints are established or maintained (Szabo P. E. and Mann J. R. Genes and Development 9 3097-3108 (1995)). These changes in DNA methylation of imprinted genes occur during normal pre-implantation embryo development in mice, at a time when virtually all other DNA in the genome is entirely demethylated (Li, E., in Genomic Imprinting, pages 1-20, ed.s Riek, W. and Surani, A., IRL Press, Oxford (1997); Kafri et al Proc. Nat'l Acad. Sci. USA 90 10558-10562 (1993)). However, no definitive cause and effect association between expression of a particular gene or genes (whether unprinted or not) and large Offspring syndrome in cattle or sheep has been identified to date.

During the period of in vitro maturation, many cattle and sheep oocytes undergo in vitro culture in medium containing a variety of sera and co-cultured cells (Trounson et al Theriogenology 41 57-66 (1994); Marquant-Leguienne, B. and Humbolt, P. Theriogenology 49 3-11 (1998); Sirard, M. A. and Blondin, P., Animal Reproduction Science 42 417-426 (1996); Thompson J. G. Reprod. Fertil. Dev. 9 341-354 (1997)), i.e. the same conditions which are associated with induction of LOS during exposure of the early embryo (Thompson et al Biology of Reproduction 53 1385-1391 (1995); Sinclair et al Theriogenology 47 380 (1997)). It is not yet clear whether the period of in vitro maturation can perturb an oocyte, in ways which induce LOS, since very few studies have addressed this question. However, some evidence is provided by the study of Holm et al (Journal of Reproduction and Fertility 107 175-181 (1996)), which indicates that the syndrome may have arisen from oocytes matured and fertilised in vitro and then replaced into recipient ewes for the embryo culture period and the remainder of gestation. For many imprinted genes, imprints and associated changes in DNA methylation are established during gametogenesis and any epigenetic alterations at this stage induced by oocyte exposure to in vitro maturation may also result in phenotypes typical of LOS, by similar mechanisms to those proposed for embryo culture. Similarly, epigenetic alterations may arise in the oocyte when subjected to in vitro culture within an ovarian follicle prior to subsequent oocyte maturation. Follicle culture has recently been suggested as a means of improving the subsequent developmental competence of bovine oocytes matured in vitro (Fouladi Nashta et al Biology of Reproduction 59 255-262 (1998)). It is also possible that, in some cases at least, abnormalities in imprinted genes intrinsic to the oocyte are responsible for initiating LOS. By using procedures such as in vitro maturation or superovulation, more oocytes are obtained from a single ovary than would normally mature in vivo in an oestrous cycle. Most of the oocytes matured by these procedures would die prior to ovulation in vivo. These procedures are often used to increase the availability of oocytes for in vitro production or nuclear transfer and may “rescue” oocytes with imprinting defects which would not normally survive in vivo. This type of “rescue” may also occur during the period of embryo culture, although it is known that exposure to the period of embryo culture alone is sufficient to induce LOS (Thompson et al Biology of Reproduction 53 1385-1391 (1995); Sinclair et al Theriogenology 47 380 (1997))

The induction of LOS subsequent to nuclear transfer is also poorly understood. For example, it has not been demonstrated whether the perturbation is due to nuclear transfer per se or to the associated periods of oocyte or embryo culture. Most nuclear transfer embryos are either cultured subsequently in vitro using protocols associated with perturbed growth and development (for example see Yazawa et al Theriogenology 48 641-650 (1997), or ligated in a sheep oviduct with no regard to synchrony of the recipient animal with the developmental stage of the reconstructed embryo (for example see Willadsen et al Theriogenology 35 161-170 (1991). Exposure of sheep embryos to an asynchronous reproductive tract environment has previously been demonstrated to result in fetal oversize (Wilmut, I. and Sales, D. I. J. Reprod. Fertil. 61 179-184 (1981); Young et al Theriogenology 45 231 (1996)). In addition to the possible asynchrony, ligation in an oviduct is in itself an unusual environment. During the period of development to the blastocyst stage (normally used for embryo transfer to gestation recipients in cattle and sheep), embryos would naturally be in the uterus for 3-4 days and exposed to uterine secretions. Ligation of the oviduct may, therefore, expose the embryo to unusual secretions which provide a perturbing milieu, resulting in LOS. In cattle and sheep embryos genetically modified by pronuclear injection, subsequent development also commonly occurs in a ligated oviduct or in in vitro embryo culture.

In addition, it has been suggested (Jaenish R. Trends in Genetics 13 323-329 (1997) that the successful use of nuclei for donors in nuclear transfer, in terms of efficiency and frequency of fetal death may in part depend on the incidence of methylation errors found in imprinted genes in the donor cell. It is argued that incidence of such errors would be related to the age of the animal from which the donor cell was derived, with older cells having more errors. This awaits direct experimental evidence but such errors specifically in imprinted genes would not be erased by the demethylation and de novo DNA methylation events which occur during early embryogenesis (Kafri et al Genes Dev. 6 705-714 (1992)), during the time of nuclear reprogramming. In some cases, these errors may provide another possible route for induction of Large Offspring syndrome.

It is believed that, in theory, any manipulation of embryos or cells used to derive embryos which may alter the environment of such cells or the genetic material of these cells, including DNA and RNA, may induce epigenetic change in a vulnerable gene or genes. Procedures involving drilling or penetration of the zona pellucida, for example during enucleation, intracytoplasmic sperm injection (Barnes et al Human Reproduction 10 3243-3247 (1995)) or pre-implantation diagnosis (Handyside, A. H. and Delhanty J. D. Trends in Genetics 13 270-275 (1997)), may expose the genetic material to factors normally excluded by the zona pellucida. Procedures such as cytoplasmic transfer or cytoplasmic reduction (Cohen et al Mol. Hum. Reprod. 4 269-280 (1998); Cohen et al Lancet 350 186-187 (1997); Eviskov et al Development 109 323-328 (1990); Eviskov et al Roux's Arch. Dev. Biol. 203 199-204 (1994)) may alter the concentration of cytoplasmic factors which influence DNA (Moore, T. and Riek, W., Rev. Reprod. 1 73-77 (1996)) or RNA. In addition, procedures such as intracytoplasmic sperm injection or nuclear transfer, which involve introduction of foreign material into a recipient oocyte or cell, may inadvertently introduce factors into the recipient which may be perturbing.

Thus there are several possible routes for initiation of LOS in cattle and sheep, all of which may act through epigenetic alterations in imprinted genes. These include epigenetic alterations induced by in vitro culture of oocytes or embryos, exposure of oocytes or embryos to an unusual environment (including a ligated oviduct), intrinsic defects in oocytes matured in vitro or in vivo by superovulation, intrinsic defects in donor cells used for nuclear transfer and inappropriate reprogramming of imprinting subsequent to nuclear transfer.

Central to the regulation of mammalian fetal growth is insulin-like growth factor II (IGFII). IGFII regulation occurs in a dynamic and hierarchical manner with controls acting at the levels of transcription, translation, protein binding or receptor interactions (Zarrilli et al Mol. Cell. Endocrinol 101 R1-R4 (1994)). The Igf2 gene is imprinted in the mouse (DeChiara et al Cell 64 849-859 (1991)), human (Giannoukakis et al Nature Genetics 4 94 (1993) and sheep (Feil et al Mammalian Genome—in press (1998); Hagemann et al Mol. Reprod. Dev. 50 154-162 (1998)) and loss of imprinting consequent to the failure of imprinting mechanisms leads to biallelic expression and therefore overexpression of the Igf2 gene. Loss of imprinting of this gene leading to fetal overgrowth in both humans and mice is thought to be caused by an increased concentration of insulin-like growth factor-II (IGFII) protein (Eggenschwiler et al Genes Dev. 11 3128-3142 (1997); Sun et al Nature 389 809-815 (1997)). In mice, loss of imprinting of Igf2 has been induced by deleting the adjacent H19 gene which appears to be involved in the Igf2 imprinting mechanism (Leighton et al Nature 375 34-39 (1995); Ohlsson et al Development 120 361-368 (1994)). Transgenic overexpression of the mouse Igf2 gene results in prenatal overgrowth and other effects which are similar to those of Large offspring syndrome in cattle and sheep and Beckwith-Weidmann syndrome in humans. These include polyhydramnios, fetal and neonatal lethality, organ and skeletal abnormalities (Sun et al Nature 389 809-815 (1997)).

In some cases of Beckwith-Weidmann syndrome in humans, fetal overgrowth results from disomies, trisomies or other chromosomal rearrangements of the H19/Igf2 region. However, in many sporadic cases of the syndrome, the observed loss of imprinting and resulting biallelic overexpression of the Igf2 gene is not associated with chromosomal abnormalities. Instead, these cases have been suggested to result from “epigenetic accidents” in the H19/Igf2 domain in the germline or early embryo (Reik et al Human Molecular Genetics 4 2379-2385 (1995)), although these have not yet been identified or described and it is not known how they might arise. Environmentally-induced epigenetic alterations to imprinted genes, such as alterations in DNA methylation, were suggested as the possible cause of prenatal and perinatal losses and large size observed in mouse fetuses derived from ES cells in in vitro culture (Nagy et al Proc. Nat'l Acad. Sci USA 90 8424-8428 (1993)). Although, it is not yet known which gene(s) is responsible for this effect, it has now recently been shown that epigenetic alterations in DNA methylation and imprinted gene expression arising from derivation or culture of mouse embryonic stem (ES) cells do not become corrected during post-implantation development and are associated with aberrant gene expression in the ES cell-derived fetus (Dean et al Development 125 2273-2282 (1998)). This study found evidence of alterations in all four imprinted genes which were examined, although these were inconsistent between and within the (four) different ES cell lines tested. In particular, loss of methylation of the maternal U2afI-rs1 allele was commonly detected and resulted in biallelic expression of this gene (which encodes a splicing factor of unknown biological function). Biallelic methylation of H19 was also common and this resulted in strongly-repressed H19 expression in the ES cell fetuses. In all ES cell lines studied, the maternal Igf2 allele was unusually methylated, in addition to the paternal allele. In several fetuses, this resulted in expression of Igf2 from both alleles, but particularly from the maternal allele. Finally, the Igf2r gene was maternally methylated in all ES cell lines and in most ES cell fetuses. In all ES cell lines the pattern of Igf2r expression was biallelic and in the majority of ES cell fetuses was only from the maternal allele. This is consistent with the patterns of Igf2r methylation and expression found in normal mouse blastocyst-stage embryos (from which ES cells are derived) and fetuses. These observations confirm previous reports of the stability of Igf2r imprinting in mouse ES cell lines (Labosky et al Development 120 3197-3204 (1994)). However, some ES fetuses derived from two ES cell lines showed partial biallelic expression of Igf2r (with <35% of the expression from the normally silent, paternal allele). In addition, in one ES fetus (out of 24), biallelic Igf2r expression was observed and in this case methylation of both parental alleles was evident.

The role of imprinting in Igf2r expression and the fact that lack of expression results in fetal overgrowth in mice has been used to hypothesise that a corresponding loss of imprinting in Igf2r (i.e. biallelic expression) would have the opposite effect to that of Igf2 and would instead lead to a reduction in fetal growth (Moore, T. and Reik, W. Rev. Reprod. 1 73-77 (1996)). No such phenotype was reported in the ES cell fetuses, neither were there any culture-induced changes in Igf2r which would be consistent with fetal overgrowth or other symptoms of Large Offspring Syndrome (Dean et al Development 125 2273-2282 (1998)). Furthermore, the different developmental abnormalities described for the ES cell-derived fetuses, including polyhydramnios, mandibular abnormalities and interstitial bleeding have not so far been related to specific imprinted genes and may of course be associated with genes not examined in the reported study. These observations of aberrant imprinted gene expression in cultured, embryo-derived ES cells, coupled with the known developmental roles of imprinted genes and (in some cases) known phenotypic effects on fetal growth, add credibility to the hypothesis that imprinted genes and their epigenetic alterations underlie the Large offspring syndrome in cattle and sheep subsequent to embryo culture in an unusual environment and/or nuclear transfer (Moore, T. and Reik, W. Rev. Reprod. 1 73-77 (1996); Walker et al Theriogenology 45 111-120 (1996); Dean et al Development 125 2273-2282 (1998)). However, they shed little light on which gene or genes are directly involved.

A screening method diagnostic of inducing LOS would be of commercial benefit in identifying and improving embryo culture, nuclear transfer and other embryo manipulation protocols associated with abnormal development. There is therefore a pressing need to be able to screen effectively for the presence of abnormal development in an in vitro culture animal embryo to allow appropriate intervention or modification of the culture conditions.

Evidence is now provided that altered expression of the Igf2r gene encoding the insulin-like growth factor II receptor (IGFIIR) is predictive of abnormal development of an animal embryo with symptoms typically associated with Large Offspring syndrome (LOS). Embryos at risk from LOS include embryos cultured in vitro or in an unusual in vivo environment, as well as reconstituted embryos prepared by nuclear transfer.

According to a first aspect of the present invention there is provided a method of screening an animal embryo for Large Offspring Syndrome comprising the step of analysing Insulin-like growth factor-2 receptor (Igf2r) gene expression in the animal embryo or in a biological sample from the embryo.

The screening of animal embryos by a method according to the present invention can avoid the problems encountered where an in vitro culture animal embryo is affected by the symptoms of Large Offspring Syndrome in which the embryo can grow to a large size or have associated organ defects or developmental abnormalities. The method is also applicable to an animal embryo cultured in an altered or an unusual environment in vivo. The use of the screening method will allow for intervention in the development of the embryo to arrest or to prevent the abnormal development. The detection of pluripotential cells and embryos or cells derived from them which are likely to result in the production of abnormal offspring would allow selection of appropriate lines for generation of chimeras or cell-derived embryos. Methods in accordance with the present invention will also permit the identification of culture conditions which do not initiate the defects leading to Large Offspring syndrome. Such methods may be of considerable commercial benefit in agricultural species and may have future applications in several other species, including humans. This may be of particular relevance to any future use of stem cells or other cells used in human cell-based therapies for medical applications (Solter, D., Nature 394 315-316 (1998)).

The screening method of the present method is generally applicable to any animal embryo, including birds, such as domestic fowl, fish, eutherian and marsupial mammals, including where the embryo is the result of nuclear transfer or in vitro production (IVP), comprising in vitro culture (IVC) and/or in vitro fertilisation (IVF) and/or in vitro maturation (IVM) of an oocyte, and further including culture in any environment in which an embryo would not find itself in or exposed to during normal gestation, e.g. in a ligated oviduct in a temporary recipient animal. However, the method may find its main use in screening mammalian embryos, particularly ruminant, human or primate embryos. Other mammalian species to which the method may find application, include but are not limited to, non-human mammals, for example, ungulate species, such as cattle, sheep, pigs, goats, horses, camels or buffalo (including water buffalo), and species such as dogs, cats, horses, llamas, alpacas and rodents including rats, mice, or rabbits, or guinea pigs, and a variety of animal (wildlife or non-domesticated) species, including pandas, tigers, and cetacean species including whales and dolphins.

The method may also find application to transgenic or genetically modified animal embryos, including chimeras and embryos prepared by nuclear transfer procedures or where the embryo has been the subject of manipulation to alter its genetic content. Such methods may also find utility in cell-based transgenics using ES cells or other cells in culture using nuclear transfer. For example, in the screening of embryos or embryo-derived cells, particularly ES or EG cells (Dinsmore et al Theriogenology 49 145-151 (1998)) which involve modifications of the animal's genome where embryonic cells are used to make specific cell types for medical therapeutic uses, including in vitro differentiation of such cells.

It should be noted that the term “transgenic”, in relation to animals, should not be taken to be limited to referring to animals containing in their genome or germ line one or more genes from another species, although many transgenic animals will contain such a gene or genes. Rather, the term refers more broadly to any animal whose germ line or genome has been the subject of technical intervention by recombinant DNA technology. So, for example, an animal in whose germ line an endogenous gene has been deleted, duplicated, activated or modified is a transgenic animal for the purposes of this invention as much as an animal to whose genome or germ line an exogenous DNA sequence has been added. In embodiments of the invention in which the animal is transgenic, the genetic modification may be undertaken using physical techniques such as microinjection into the male or female pronucleus of the zygote or into the cytoplasm or nucleus of an oocyte or embryo. Alternatively, the genetic modification can involve the use of mass transformation or transfection techniques such as electroporation, viral transfection (including the use of adenoviruses, retroviruses, adeno-associated means or synthetic retrotransposons), lipofection, microprojectile cell bombardment, antisense technology, vectors such as YAC and BAC or by using other means such as sperm. Furthermore the modification can benefit from intervention by homologous recombination, DNA repair mechanisms, including the use of restriction enzymes. Cell-mediated transgenesis can employ a variety of cells, including ES cells, EG cells and other stem cells or suitable cells from any mammalian species.

Further applications may be found using nuclear transfer of non-genetically modified animals. For example unmodified clones may be used to multiply genetically superior or useful individuals for agricultural applications, including increased milk and beef yield or quality and increased reproductive performance. It may also be used in situations where endangered species are cloned to maintain a breed, population or species (e.g. tiger, rhino, panda, whale or dolphin). In addition, screening of an embryo for potential development of LOS-associated abnormalities may also be of immense benefit in the potential development and use of pre-implantation human embryos for cell-based therapies or of other embryos for transplantation.

Methods according to the present invention are employed to screen an animal embryo. The culture of embryos in vitro is now a well established technique in in vitro production of animals, including in vitro maturation of oocytes. It has found application in overcoming infertility problems in humans and in controlled breeding in agricultural farm animals commonly using in vitro fertilisation where an oocyte and sperm are brought together in a controlled and observable environment outside the body. In vitro culture of animal embryos also includes animal embryos prepared by nuclear transfer procedures or animal embryos which have been the subject of manipulation to alter their genetic content. In vivo culture of embryos is also commonly employed, especially after reconstruction of an embryo by nuclear transfer or genetic modification by pronuclear injection. This generally involves surgical ligation of the embryos in the oviduct of a recipient animal, where the embryos may be embedded in a protective, permeable but inert medium, e.g. agar or an equivalent substance (Campbell et al Biol. Reprod. 50 1385-1393 (1994)). The recipient animal is not always of the same species as the in vivo cultured animal embryo (Wilson et al Anim. Reprod. Soc. 38 73-83 (1995)). The method is also applicable to the screening of any animal embryo cultured in an altered or unusual environment which may result in the development of Large Offspring Syndrome (LOS), comprising any environment in which an embryo would not find itself in or be exposed to during normal gestation. This may include transfer of the embryo from the reproductive tract from one animal to another, or to another part of the reproductive tract of the same animal. It may also include direct or indirect alterations to the reproductive tract environment. Examples of direct alterations include, but are not limited to, flushing the tract with a suitable fluid or addition of any implant, foreign substance or object. Indirect alterations may include, but are not limited to, dietary, endocrine or drug administration to the recipient animal.

The term “embryo” is used to describe the developing animal following conception and the first division of the zygote until the birth of the new-born animal. The term therefore includes more specialised descriptive terms “blastula”, “gastrula”, and “foetus” (or “fetus”). Fetus can be used to describe an embryo when the first bone cells appear in the cartilage, after implantation. The term also includes “blastocyst” which describes the stage of development in humans at which implantation into the uterine wall occurs in normal gestation which occurs when the inner cell mass (ICM) spreads inside the blastocoele as a flat disc. In other animal species, such as sheep and cattle, implantation may not occur until after the blastocyst stage and so this definition should be understood to extend to the stage of development which consists of inner cell mass cells and trophectoderm cells around a central blastocoele cavity.

The method of the present invention is directed to screening for Large Offspring Syndrome (LOS). The syndrome is most commonly seen in agricultural farm animals produced by nuclear transfer procedures, in vitro genetic manipulation and/or in vitro production techniques, including the use of in vitro maturation, in vitro fertilisation and/or in vitro embryo culture. The phenomenon of Large Offspring Syndrome is often difficult to characterise as it is not only exhibited in terms of birthweight. There are also other associated abnormalities including increased rates of abortion and physical abnormalities, increased gestation length and increased levels of mortality and morbidity (Walker et al Theriogenology 45 111-120 (1996)). The abnormal development is with respect to that of a normal animal embryo at the equivalent stage of development.

The presence of Large Offspring Syndrome (LOS) in an embryo screened using a method according to the present invention is diagnosed following an analysis of the expression of the gene coding for the insulin-like growth factor-2 receptor (IGF2R) in the animal embryo. The IGF2R is also sometimes referred to as the mannose 6-phosphate receptor, the Type II IGF receptor and the cation-independent mannose 6-phosphate receptor. References to IGF2R protein and Igf2r gene should therefore be understood to include these definitions. The altered or perturbed expression of the Igf2r gene is indicative of abnormal development in the animal embryo compared to normal levels of gene expression. The altered gene expression may be seen as a reduced level of transcription of the DNA encoding the IGF2R. This change may also be demonstrated by an altered pattern of DNA methylation in the gene coding for IGF2R in the affected embryo. Other changes in DNA, including alterations in chromatin structure or histone acetylation may also be associated with altered Igf2r expression. The change may also be indicated by a reduction in the levels of IGF2R protein compared to normal embryos of the same gestational age. Where the expression of Igf2r in the animal is reduced this is predictive of the embryo being at risk of developing Large Offspring Syndrome (LOS). Without wishing to be bound by theory, it is believed that the reduction in the expression of the Igf2r gene is accompanied by no alteration in the expression of the Igf2 gene. It will also be appreciated that a method of the present invention may also find use in screening a neonate or new-born animal as some features of LOS are not readily apparent and there may be circumstances in which a screen at this stage shortly after birth would be useful.

The analysis of gene expression is carried out on a biological sample from the embryo. The sample may comprise a tissue biopsy, cells or cellular material or a biological fluid, such as for example, blood, taken from the embryo, or amniotic or allantoic fluid from the placenta. The tissue or cell sample may be taken from an organ, muscle, cartilage, bone or skin. However, in the early stage embryo it is likely that the sample will conveniently be taken from a cell of the developing embryo. In some cases, the entire embryo may be screened. The sample may also be an oocyte or an ovarian follicle which incorporates an oocyte at any stage of development from the oogonium, through the primary oocyte stage to the secondary oocyte stage and the mature ovum.

Analysis of Igf2r gene expression may be achieved by any standard means including PCR based methods, e.g. RT-PCR. Igf2r primers can be designed from Genbank bovine sequences, for example sequence J03527 (5′-nucleotides 530-551, 3′-nucleotides 769-750; Lobel et al J. Biol. Chem. 263 (5) 2563-2570 (1988)).

In some cases, a comparison for control purposes can be made in a method of the invention between levels of expression of Igf2 and Igf2r. Primers for Igf2 exon-specific transcripts can be obtained as described by Ohlsen et al in DNA Cell Biol. 13 (4) 377-388 (1994), IGFBP2, IGFBP3 as described in Winger et al Biol. Reprod. 56 1415-1423 (1997) and IGFBP4 as described in Armstrong et al Endocrinology 139 (4) 2146-2154 (1998). Igf2 is expressed as a series of alternatively-spliced, exon-specific transcripts in a tissue-specific and developmental stage-specific manner (Ohlsen et al DNA Cell Biol. 13 (4) 377-388 (1994)).

A method of the present invention can therefore be considered to comprise the use of a nucleic acid sequence encoding IGF2R or a fragment thereof or a sequence complementary or homologous thereto in the preparation of an agent for the diagnosis of Large Offspring Syndrome (LOS) in an animal embryo or in a sample from the embryo. The sequence of the gene Igf2r encoding IGF2R is known in humans (Oshima et al J. Biol. Chem. 263 (5) 2553-2562 (1988); Morgan et al Nature 329 301-307 (1987)), in bovines (Lobel et al J. Biol. Chem. 263 (5) 2563-2570 (1988)) and in mice (Ludwig et al Gene 142 (2) 311-312 (1994)) and the skilled person can select appropriate primer sequences based on standard molecular biological techniques (Sambrook et al Molecular Cloning: A Laboratory Manual, Second edition (1989)). Such nucleic acid sequences may be used directly, or more commonly they will be used to design appropriate primers for use in PCR based assays.

A nucleic acid sequence which is complementary to a nucleic acid sequence useful in a method of the present invention is a sequence which hybridises to such a sequence under stringent conditions, or a nucleic acid sequence which is homologous to or would hybridise under stringent conditions to such a sequence but for the degeneracy of the genetic code, or an oligonucleotide sequence specific for any such sequence. The nucleic acid sequences include oligonucleotides composed of nucleotides and also those composed of peptide nucleic acids. Where the nucleic sequence is based on a fragment of the gene encoding IGF2R, the fragment may be at least any ten consecutive nucleotides from the gene, or for example an oligonucleotide composed of from 20, 30, 40, or 50 nucleotides.

Stringent conditions of hybridisation may be characterised by low salt concentrations or high temperature conditions. For example, highly stringent conditions can be defined as being hybridisation to DNA bound to a solid support in 0.5M NaHPO₄, 7% sodium dodecyl sulfate (SDS), 1 mM EDTA at 65° C., and washing in 0.1×SSC/0.1% SDS at 68° C. (Ausubel et al eds. “Current Protocols in Molecular Biology” 1, page 2.10.3, published by Green Publishing Associates, Inc. and John Wiley & Sons, Inc., New York, (1989)). In some circumstances less stringent conditions may be required. As used in the present application moderately stringent conditions can be defined as comprising washing in 0.2×SSC/0.1% SDS at 42° C. (Ausubel et al (1989) supra). Hybridisation can also be made more stringent by the addition of increasing amounts of formamide to destabilise the hybrid nucleic acid duplex. Thus particular hybridisation conditions can readily be manipulated, and will generally be selected according to the desired results. In general, convenient hybridisation temperatures in the presence of 50% formamide are 42° C. for a probe which is 95 to 100% homologous to the target DNA, 37° C. for 90 to 95% homology, and 32° C. for 70 to 90% homology.

Examples of preferred nucleic acid sequences for use in a method of the present invention are oligonucleotide primers based on 5′-nucleotides 530-551, 3′-nucleotides 769-750 of the bovine sequence of the Igf2r gene (Lobel et al J. Biol. Chem. 263 (5) 2563-2570 (1988)).

Methods in accordance with the present invention have advantages over the techniques for in vitro production (including in vitro fertilisation and in vitro maturation), genetic modification and nuclear transfer previously described. The ability to screen for abnormal development should permit the efficiency of the techniques to be enhanced by reducing the number of unsuccessful pregnancies where an embryo develops abnormally and dies during embryonic development, or at birth or shortly thereafter. The identification of embryos at risk from developing abnormally should also permit measures to be taken to safeguard the health of the female carrying the embryo.

According to a second aspect of the present invention there is provided a method for in vitro production of an animal embryo comprising the steps of introducing a sperm cell to an oocyte in an in vitro culture system, subsequently culturing the resulting embryo and screening the embryo for Large Offspring Syndrome (LOS) by analysing Igf2r gene expression in a biological sample from the embryo.

Generally, the oocyte to be fertilised will be subjected to in vitro maturation step prior to being brought into proximity with the sperm cell. In vitro production of an animal embryo will typically involve in vitro fertilisation of animal oocytes and can be achieved by any suitable method depending upon the animal species. Examples of such methods include, but are not limited to those of Trounson et al Theriogenology 41 57-66 (1994); Thompson, J. G. Reprod. Fertil. Dev. 9 341-354 (1997); Wilmut et al in Genetics of the Sheep, pages 395-412 ed.s Piper, L. and Ruvinsky, A., CAB International, Oxford, UK (1997); Gardner, D. K. and Lane, M, Human Reproduction Update 3 367-382 (1997); Summers et al Biol. Reprod. 53 431-437 (1995); Weston, A. M. and Wolf, D. P. Mol. Reprod. Dev. 44 88-92 (1996); Liu et al Mol. Reprod. Dev. 45 157-162 (1996); Li et al Theriogenology 47 1103-1113 (1997); Biggers et al Human Reproduction Update 3 125-135 (1997); Trounson, A. and Gardner, D. K. ed.s Handbook of in vitro Fertilisation, CRC Press Inc. Salem, USA (1993)). This aspect of the invention may also involve in vitro culture of the resultant animal embryos. It will also be readily appreciated that a method according to this aspect of the invention may also include further screening steps for the viability of the embryo and its suitability for transfer to the final female recipient. Additionally, the method also extends to an embryo which is the result of normal fertilisation but which is subsequently removed from the female animal and cultured in vitro for a period of time prior to re-implantation in a receptive female animal.

Oocytes suitable for in vitro maturation are commonly derived from either aspirating fresh abattoir ovaries, or from donor animals or patients, using surgical laparoscopy or ultrasound-guided “ovum pick-up” (OPU) (Wilmut et al in Genetics of the Sheep, pages 395-412 ed.s Piper, L. and Ruvinsky, A., CAB International, Oxford, UK (1997); Trounson, A. and Gardner, D. K. ed.s Handbook of in vitro Fertilisation, CRC Press Inc. Salem, USA (1993)). In some cases the donors may have been pre-treated with hormones or by other agents. In vitro maturation is most commonly performed in tissue culture medium 199 with a variety of supplements, including co-cultured support cells, gonadotrophic hormones, oestradiol, growth factors, pyruvate, serum and polyvinyl alcohol (Thompson, J. G. Reprod. Fertil. Dev. 9 341-354 (1997): Barnes et al Human Reproduction 10 3423-3247 (1995)) which allows development of an oocyte to the stage at which it may be fertilised.

In vitro fertilisation can occur in any conditions which support both motility and capacitation of spermatozoa, yet also maintain the viability of the oocytes to be fertilised. These conditions vary widely between species. As distinct from ruminants, for example, human sperm do not require any particular induction of capacitation (Trounson et al Theriogenology 41 57-66 (1994)). For bovine oocytes, the most commonly used fertilisation medium is Tyrode's albumin lactate pyruvate medium (TALP) usually supplemented with several capacitating and motility stimulating agents such as heparin, epinephrine, hypotaurine and penicillamine (Thompson. J. G. Reprod. Fertil. Dev. 9 341-354 (1997)). In contrast to the cow, simple media such as synthetic oviduct fluid (SOF) are routinely used for sheep in vitro fertilisation. Motile sperm are prepared by several methods, including “swimming-up” and using Percoll gradients (Wilmut et al in Genetics of the Sheep, pages 395-412 ed.s Piper, L. and Ruvinsky, A., CAB International, Oxford, UK (1997)).

In vitro methods for the culture of embryos produced from either in vitro maturation and/or fertilisation protocols or from superovulation regimes and donor embryo recovery vary widely both between and within species. There are essentially two culture systems: co-culture (or medium previously “conditioned” on cells) and defined (or semi-defined). Co-culture of cattle and sheep embryos is normally performed in either tissue culture medium 199 or Menezo's B2 medium, usually supplemented with serum. The support cells are usually bovine granulosa, bovine oviduct epithelial or buffalo rat liver cells. Embryo culture systems without somatic cell support are commonly referred to as semi-defined or defined systems, depending on whether sera (or a protein source such as bovine serum albumin) or synthetic macromolecule (such as polyvinyl alcohol) supplementation is used. Widely-used defined media include synthetic oviduct fluid (SOF) and CR1, to which a wide variety of sera and other supplements (such as growth factors, hormones, vitamins, amino acids, enzymes and antioxidants) may be added (Thompson, J. G. Reprod. Fertil. Dev. 9 341-354 (1997)). Embryos have also been cultured in bodily fluids or media which contain bodily fluids such as follicular fluid and human amniotic fluid (Trounson et al Theriogenology 41 57-66 (1994)). In addition to static embryo culture systems, the use of perfusion systems for continuous delivery of fresh culture medium are being developed (Thompson, J. G. Theriogenology 45 27-40 (1996)).

Cattle and sheep embryos are routinely cultured to the blastocyst stage (after 5-7 days in embryo culture) prior to transfer into a suitable recipient animal while human embryos are generally transferred to the uterus on day 2 or 3 of development. However, culture of human embryos to the blastocyst stage is becoming more widely contemplated (Gardner, D. K. and Lane, M, Human Reproduction Update 3 367-382 (1997)) or at least up until development of the primitive streak. The primitive streak can be taken to have appeared in an embryo not later than the end of the period of 14 days beginning with the day the gametes are mixed, not counting any time during which the embryo is stored. In vitro embryo culture is required as part of many current and potential applications as well as for assisted reproduction in humans and other species. Many genetically-manipulated embryos produced by pronuclear injection have been cultured prior to transfer into a recipient, as have many embryos reconstructed by nuclear transfer. In vitro embryo culture has been used in protocols for the derivation of embryonic stem cells (ES cells) and ES cell-like cells and may be important in the development of cell-based therapies for clinical use. Despite a considerable research effort and great improvement in effectiveness of the techniques, there is still not a method of producing embryos at a comparable rate to in vivo (Wilmut et al in Genetics of the Sheep, pages 395-412 ed.s Piper, L. and Ruvinsky, A., CAB International, Oxford, UK (1997)).

According to a third aspect of the invention there is provided a method for reconstituting an animal embryo comprising the step of transferring a nucleus from a donor cell into a suitable recipient cell and screening the resulting embryo for Large Offspring Syndrome (LOS) by analysing Igf2r gene expression in a biological sample from the embryo.

In the method of the invention described in the above aspect, a nucleus is transferred from a donor cell to a recipient cell. The use of this method is not restricted to a particular donor cell type. The donor cell may be as described in Wilmut et al Nature 385 810 (1997); Campbell et al Nature 380 64-66 (1996); Cibelli et al Science 280 1256-1258 (1998); or Wakayama et al Nature 394 369-373 (1998)). All cells of normal karyotype, including embryonic, foetal and adult somatic cells which can be used successfully in nuclear transfer may in principle be employed in a method according to the present invention. Foetal fibroblasts are a particularly useful class of donor cells. Generally suitable methods of nuclear transfer are described in Campbell et al Theriogenology 43 181 (1995), Collas et al Mol. Reprod. Dev. 38 264-267 (1994), Keefer et al Biol. Reprod. 50 935-939 (1994), Sims et al Proc. Nat'l. Acad. Sci. USA 90 6143-6147 (1993), Wakayama et al Nature 394 369-373 (1998), WO-A-9426884, WO-A-9424274, WO-A-9807841, WO-A-9827214, WO-A-9003432, U.S. Pat. No. 4,994,384 and U.S. Pat. No. 5,057,420. The invention therefore contemplates the use of an at least partially differentiated cell, including a fully differentiated cell. Donor cells may be, but do not have to be, in culture and may be quiescent. Nuclear donor cells which are quiescent are cells which can be induced to enter quiescence or exist in a quiescent state in vivo. Cultured bovine primary fibroblasts, an embryo-derived ovine cell line (TNT4), an ovine mammary epithelial cell derived cell line (OME) from a 6 year old adult sheep, a fibroblast cell line derived from foetal ovine tissue (BLWF1) and an epithelial-like cell line derived from a 9day old sheep embryo (SEC1) are described in WO-A-9707669 and WO-A-9707668. A class of embryo-derived cell lines useful in the invention which includes the TNT4 cell line described in WO-A-9607732. Cultured inner cell mass (CICM) cells are described in WO-A-9737009 and WO-A-9827214 and embryonic or stem-like cell lines are described in WO-A-9807841. Transgenic bovine fibroblasts for use as nuclear donors are described in Zawada et al (Nature Medicine 4 (5) 569-574 (1998) and in Cibelli et al (Science 280 1256-1258 (1998)).

Where the donor cells are described as being quiescent, such cells may not be actively proliferating by means of the mitotic cell cycle. The use of a quiescent donor cell is described in WO-A-9707669. The mitotic cell cycle has four distinct phases, G1, S, G2 and M. The beginning event in the cell cycle, called start, takes place in the G1 phase and has a unique function. The decision or commitment to undergo another cell cycle is made at start. Once a cell has passed through start, it passes through the remainder of the G1 phase, which is the pre-DNA synthesis phase. The second stage, the S phase, is when DNA synthesis takes place. This is followed by the G2 phase, which is the period between DNA synthesis and mitosis. Mitosis itself occurs at the M phase. Quiescent cells (which include cells in which quiescence has been induced as well as those cells which are naturally quiescent, such as certain fully differentiated cells, e.g. Sertoli cells, neurons or cumulus cells) are generally regarded as not being in any of these four phases of the cycle; they are usually described as being in a G0 state, so as to indicate that they would not normally progress through the cycle, or are not actively dividing by means of the cell cycle. The nuclei of quiescent G0 cells have a diploid DNA content.

Cultured cells can be induced to enter the quiescent state by various methods including chemical treatments, nutrient deprivation, growth inhibition or manipulation of gene or protein expression. Presently the reduction of serum levels in the culture medium has been used successfully to induce quiescence in both ovine and bovine cell lines. In this situation, the cells exit the growth cycle during the G1 phase and arrest, as explained above, in the so-called G0 stage. Such cells can remain in this state for several days (possibly longer depending upon the cell) until re-stimulated when they re-enter the growth cycle. Quiescent cells arrested in the G0 state are diploid. The G0 state is the point in the cell cycle from which cells are able to differentiate. On quiescence a number of metabolic changes have been reported and these include: monophosphorylated histones, ciliated centrioles, reduction or complete cessation in all protein synthesis, increased proteolysis, decrease in transcription and increased turnover of RNA resulting in a reduction in total cell RNA, disaagregation of polyribosomes, accumulation of inactive 80S ribosomes and chromatin condensation (reviewed Whitfield et al., Control of Animal Cell Proliferation, 1 331-365 (1985)).

Many of these features are those which are required to occur following transfer of a nucleus to an enucleated oocyte. The fact that the G0 state is associated with cell differentiation suggests that this may provide a nuclear/chromatin structure which is more amenable to either remodelling and/or reprogramming by the recipient cell cytoplasm. In this way, by virtue of the nuclear donor cells being in the quiescent state, the chromatin of the nuclei of the donors may be modified before embryo reconstitution or reconstruction such that the nuclei are able to direct development. This differs from all previously reported methods of nuclear transfer in that the chromatin of donor cells is modified prior to the use of the cells as nuclear donors.

The recipient cell to which the nucleus from the donor cell is transferred may be an oocyte or another suitable cell. Reprogramming of cell other than oocytes has been demonstrated, including embryonic stem cells (Matveeva et al Mol. Reprod. Dev. 50 128-138 (1998) and embryonic germ cells (Tada et al EMBO J. 16 6510-6520 (1997)).

Recipient cells at a variety of different stages of development may be used, from oocytes at metaphase I through metaphase II, to zygotes and two-cell embryos (Cheong et al Jpn. J. Vet. Res. 40 149-150 (1992)). Each has its advantages and disadvantages. The use of fertilised eggs ensures efficient activation whereas parthenogenetic activation is required with oocytes (see below). Another mechanism that may favour the use of cleavage-stage embryos in some species is the extent to which reprogramming of gene expression is required. Transcription is initiated during the second cell cycle in the mouse and no major changes in the nature of the proteins being synthesised are revealed by two-dimensional electrophoresis until the blastocyst stage (Howlett & Bolton J. Embryol. Exp. Morphol. 87 175-206 (1985)). In most cases, though, the recipient cells will be oocytes. In cattle and sheep embryos, transcription is initiated later, during the fourth cell cycle, and so later cleavage-stage embryos may be suitable in these species. In most cases though, the recipient cells will be oocytes.

It is preferred that the recipient be enucleate. While it has been generally assumed that enucleation of recipient oocytes in nuclear transfer procedures is essential, there is no published experimental confirmation of this judgement. The original procedure described for ungulates involved splitting the cell into two halves, one of which was likely to be enucleated (Willadsen Nature 320 (6) 63-65 (1986)). This procedure has the disadvantage that the other unknown half will still have the metaphase apparatus and that the reduction in volume of the cytoplasm is believed to accelerate the pattern of differentiation of the new embryo (Eviskov et al., Development 109 322-328 (1990)).

More recently, different procedures have been used in attempts to remove the chromosomes with a minimum of cytoplasm. Aspiration of the first polar body and neighbouring cytoplasm was found to remove the metaphase II apparatus in 67% of sheep oocytes (Smith & Wilmut Biol. Reprod. 40 1027-1035 (1989)). Only with the use of DNA-specific fluorochrome (Hoechst 33342) was a method provided by which enucleation would be guaranteed with the minimum reduction in cytoplasmic volume (Tsunoda et al., J. Reprod. Fertil. 82 173 (1988)). In livestock species, this is probably the method of routine use at present (Prather & First J. Reprod. Fertil. Suppl. 41 125 (1990), Westhusin et al., Biol. Reprod. (Suppl.) 42 176 (1990)).

There have been very few reports of non-invasive approaches to enucleation in mammals, whereas in amphibians, irradiation with ultraviolet light is used as a routine procedure (Gurdon Q. J. Microsc. Soc. 101 299-311 (1960)). There are no detailed reports of the use of this approach in mammals, although during the use of DNA-specific fluorochrome it was noted that exposure of mouse oocytes to ultraviolet light for more than 30 seconds reduced the developmental potential of the cell (Tsunoda et al., J. Reprod. Fertil. 82 173 (1988)).

Transfer of the donor nucleus into the recipient oocyte may be by any suitable procedure known in the art. Techniques, include but are not limited to, fusion or injection, i.e. microinjection as described in Wilmut et al Nature 385 810 (1997); Campbell et al Nature 380 64-66 (1996); Cibelli et al Science 280 1256-1258 (1998); or Wakayama et al Nature 394 369-373 (1998)).

The reconstituted animal embryo may be cultured in vitro under suitable conditions prior to transfer into a recipient animal. Culture may up to and including any convenient stage in embryo development, e.g. to the morula, gastrula or blastocyst stage, or to a defined number of cells, e.g. 16-cell, 32-cell or 64-cell. Alternatively, culture may be up to development of the primitive streak. The primitive streak can be taken to have appeared in a human embryo not later than the end of the period of 14 days beginning with the day when nuclear transfer was carried out not counting any time during which the embryo is stored. The process may also involve temporary embryo culture in vivo. Experience suggests that embryos derived by nuclear transfer are different from normal embryos and sometimes benefit from or even require culture conditions in vivo other than those in which embryos are usually cultured (at least in vivo). The reason for this is not known. In routine multiplication of bovine embryos, for example, reconstituted embryos (many of them at once) have been cultured in sheep oviducts for 5 to 6 days (as described by Willadsen, in Mammalian Egg Transfer (Adams, E. E., ed.) 185 CRC Press. Boca Raton, Fla. (1982)). In order to protect a embryo derived from nuclear transfer or in vitro fertilisation procedures, it may be necessary to embed the embryo in a protective medium such as agar before implantation and then dissected from the agar after recovery from the temporary recipient. The function of the protective agar or other medium is twofold: first, it acts as a structural aid for the embryo by holding the zona pellucida together; and secondly it acts as a barrier to cells of the recipient animal's immune system.

If development up to the blastocyst stage has taken place in vitro, then transfer into the final recipient animal takes place at this stage. If, however, blastocyst development has taken place in vivo, although in principle the blastocyst can be allowed to develop to term in the pre-blastocyst host, in practice the blastocyst will usually be removed from the (temporary) pre-blastocyst recipient and, after dissection from the protective medium, will be transferred to the (permanent) post-blastocyst recipient to allow development to term. Culture of a large number of embryos in a single temporary recipient reduces the numbers of such animals required but a smaller number of embryos suitable to establish a successful pregnancy in the relevant species is usually transferred to permanent recipients.

According to a fourth aspect of the invention there is provided a method of reconstituting an animal embryo, the process comprising transferring a diploid nucleus into an oocyte which is arrested in the metaphase of the second meiotic division without concomitantly activating the oocyte, keeping the nucleus exposed to the cytoplasm of the recipient for a period of time sufficient for the embryo to become capable of giving rise to a live birth and subsequently activating the reconstituted embryo while maintaining correct ploidy, including screening the embryo for Large Offspring Syndrome (LOS) by analysing Igf2r gene expression in the embryo or in a biological sample from the embryo. Recipient oocytes according to this aspect of the invention are fully described in WO-A-9707668. Other features are as described previously above.

According to a fifth aspect of the present invention there is provided a reconstituted animal embryo prepared by a method as described previously in the third or fourth aspects of the invention or an animal embryo prepared by in vitro production as described in accordance with the second aspect of the invention.

According to a sixth aspect of the present invention there is provided a method for preparing an animal, the method comprising:

(a) culturing an animal embryo in vitro or in vivo environments including where the embryo has been prepared by in vitro techniques such as maturation, fertilisation and/or culture, where the embryo has been subjected to pronuclear injection, or by reconstituting an animal embryo as described above;

(b) screening the embryo for Large Offspring Syndrome (LOS) by analysing Igf2r gene expression in the embryo or in a biological sample from the embryo;

(c) causing an animal to develop to term from the embryo; and

(d) optionally breeding from the animal so formed.

Steps (a) and (b) have been described in depth above. The third step (c), in this method of the present invention is to cause an animal to develop to term from the embryo. This may be done directly or indirectly. In direct development, the reconstituted embryo from step (b) is simply allowed to develop without further intervention beyond any that may be necessary to allow the development to take place. In indirect development, however, the embryo may be manipulated before full development takes place. For example, the embryo may be split and the cells clonally expanded, for the purpose of improving yield. Alternatively, or additionally, it may be possible for increased yields of viable embryos to be achieved by means of any aspect of the present invention by clonal expansion or by serial nuclear transfer. Serial nuclear transfer may be useful if a transgenic or chimeric animal embryo is to be prepared by nuclear transfer where more than one transgene is to be inserted into the animal embryo's DNA content For example, after nuclear transfer from a donor cell (which may itself be transgenic) and embryo reconstitution, the resulting embryo can be split and its cells subject to insertion of a further transgene or transgenes to be used as a donor cell in further nuclear transfer. At each stage in the process, the embryo can be screened by a method according to the present invention to check for abnormal development. In optional step (d) of this aspect of the invention, animals may be bred from the animal prepared by the preceding steps In this way an animal may be used to establish a herd or a flock of animals having the desired genetic characteristic(s). Where an animal is transgenic, further screening of the reconstituted embryo may be carried out to select for the stable integration of the transgene(s) and correct genotype/phenotype.

By way of illustration and summary, the following scheme sets out a typical process by which transgenic and non-transgenic animals may be prepared. The process can be regarded as involving four steps:

(1) preparation of in vitro culture embryo by nuclear transfer or in vitro production;

(2) screening the embryo for Large Offspring Syndrome (LOS) by analysing Igf2r gene expression in a biological sample from the embryo;

(3) select embryos which show normal patterns of Igf2r gene expression

(4) transfer embryo to recipient female animal.

According to a seventh aspect of the invention, there is provided an animal prepared as described above.

According, to an eighth aspect of the present invention there is provided a method of screening an animal cell for Large Offspring Syndrome comprising the step of analysing Igf2r gene expression in the animal cell.

A screening method as described above allows the identification of animal cells which are at risk from contributing to the development of Large Offspring Syndrome when they are used in the in vitro production of an animal embryo or in a nuclear transfer procedure. The animal cell may obtained from an in vitro or an in vivo source.

Cells which can be screened in accordance with this aspect of the present invention include any animal cell which may be cultured in vitro. The definition includes a reconstituted single-celled embryo or a zygote. For example, embryonic stem cells (ES cells), embryonic germ cells (EG cells), sperm derived stem cells or other stem cells derived from an embryo, neonate, infant, adolescent or adult animal may be screened. In the context of the present invention, the animal stem cells may be pluripotent stem cells, embryonic stem (ES) cells, embryonic germ (EG) cells (primordial germ cell-derived or PGC-derived cells), somatic stem/progenitor cells, haematopoietic stem cells, epidermal stem cells or neuronal stem cells. A totipotent cell can direct the development of a whole animal (when constructing embryos by nuclear transfer from a donor cell into a recipient cell, such as an enucleated oocyte, it is the nucleus of the donor cell which is totipotent). This includes directing the development of extra-embryonic lineages, i.e. the placenta. In this definition, a fertilised zygote and in some species individual blastomeres are also totipotent. In contradistinction, a pluripotent or multipotent cell (i.e. an embryonic stem cell) type has been defined as one which can form all tissues in the conceptus/offspring after injection into the blastocoele cavity. In a preferred embodiment of the present invention, the animal stem cells may be embryonic stem (ES) cells or embryonic germ (EG) cells. In this definition, the terms embryonic stem (ES) and embryonic germ (EG) cells are not limited to those derived from mice but relate to any equivalent cell, such as those described as embryonic stem cell-like cells in other species.

Other cells which may be usefully screened by a method of this aspect of the invention include any cell used in or prepared by nuclear transfer or embryo reconstruction using tetraploid aggregation, blastocyst injection etc., or a cell which is the result of serial nuclear transfer. Typically such cell types include, but are not limited to, differentiated or undifferentiated cells which may also be transgenic, suitably fibroblasts. Undifferentiated cells may further be induced to differentiate by standard techniques known in the art.

It will be appreciated that a particular application of a method according to the first aspect of the invention will be in the testing of culture environments, including culture media and conditions, in vivo environments and embryo manipulation procedures, to determine whether an animal embryo cultured in such a system might be at risk from developing Large Offspring Syndrome (LOS). According to a ninth aspect of the present invention there is provided a method of testing an embryo culture environment for the induction of Large Offspring Syndrome in an animal embryo comprising the step of analysing Igf2r gene expression in a biological sample from the embryo.

A method of testing for the ability of an embryo culture environment to induce LOS in an animal embryo is especially advantageous in providing the means to determine which features of the system are responsible for the perturbation in the development of an animal embryo in embryo culture. In this regard, it is likely to find use in optimising embryo culture protocols for commercial application. This aspect of the present invention therefore also extends to the use of a method in accordance with the first aspect of the invention to test an embryo culture environment for the induction of Large Offspring Syndrome in an animal embryo.

An embryo culture environment can include embryo manipulation procedures, embryo culture procedures and nuclear transfer procedures. Typically, embryo manipulation procedures involve drilling or penetration of the zona pellucida to assist in blastocyst hatching, for sperm introduction techniques such as ICSI (intracytoplasmic sperm injection; Barnes et al Human Reproduction 10 3243-3247 (1995)), for cytoplasmic reduction or replacement (Cohen et al Mol. Hum. Reprod. 4 269-280 (1998)); Cohen et al Lancet 350 186-187 (1997)), for enucleation, transfer or removal of nuclei, pronuclei, mitochondria or any genetic material and for pre-implantation diagnosis (Handyside, A. H. and Delhanty, J. D. Trends in Genetics 13 270-275 (1997)). Other techniques of embryo manipulation include transfer of an embryo from the reproductive tract of one animal to another or to another part of the same reproductive tract. In some procedures, the oviduct is ligated with or without the embryos in agar blocks or an equivalent. The embryo's environment may also be modified by flushing or addition of a foreign substance or implant, e.g. hormones, dietary supplements or any change in the maternal environment. Embryo culture procedures include methods for the preparation and maintenance of stem cells or any other cells used for nuclear transfer or embryo reconstruction using methods such as tetraploid aggregation and blastocyst injection. Culture procedures may also include co-culture or the use of perfusion systems as well as static culture. Nuclear transfer procedures and pro-nuclear or intracytoplasmic injection of DNA are described above. Embryo culture media are well known in the art and typically contain nutrients, serum, growth factors, hormones, vitamins, anti-oxidants, enzymes and other components. The culture media may also include body fluids, e.g. follicular fluid.

The results of a method in accordance with this aspect of the invention will enable the skilled person to alter the conditions of the embryo culture environment to prevent the induction of LOS by adding or removing one or more components, or altering the concentration of a component. For example, changes in temperature, pH or gas environment; changes in levels of components such as methylating or demethylating agents, e.g. methyl transferases, 5-azacytidine, polyamines etc; changes in the expression of the Igf2r gene or the IGFIIR protein by genetic or chemical modifications, e.g. transgenesis or use of antisense oligonucleotides; addition of enzymic or other scavengers of toxic substances e.g. use of glutamate dehydrogenase to remove ammonia (Gardner, D. K. Cell Biol. Int. 18 1163-1179)).

The use of such a screening method will enable the identification of the factor(s) which act on embryos, cells and genetic material, including DNA or RNA, to perturb the development of an animal embryo by affecting expression of the Igf2r gene. This will allow the design of new or modified in vitro culture systems, embryo environments, including temporary in vivo systems, and nuclear transfer procedures which avoid the syndrome. It may also be possible to use the screen to determine the stage in development at which the perturbation of growth occurs which may provide for alternative means for changing the culture protocol.

Embryos which have been screened according to a method of the present invention or prepared by a method which incorporates a step for screening for LOS can be used as sources of additional nuclear donor cells as described above. The embryos can be further utilised as a source of cells to produce a cell line which may be an ES cell or ES cell-like cell. Animal cells or cell lines derived from such embryos could also be used in cell-transplantation therapy. Accordingly, there is provided in a further aspect of the invention a method of therapy comprising the administration of animal cells to a patient, wherein the cells have been prepared from an embryo screened according to a method as described above, or which has been prepared according to a method incorporating such a screening step. This aspect of the invention extends to the use of such cells in medicine, e.g. cell-transplantation therapy, and also to the use of cells derived from such embryos in the preparation of a cell or tissue graft for transplantation. The cells may be organised into tissues, for example, heart, lung, liver, kidney, pancreas, corneas, nervous (e.g. brain, central nervous system, spinal cord), skin, or the cells may be blood cells (e.g. haemocytes, i.e. red blood cells, leucocytes) or haematopoietic stem cells or other stem cells (e.g. bone marrow). For example, an autograft may be prepared where the cells are removed from the patient prior to modification and returned subsequently. However, a method of the present invention will also find utility in the screening of embryos in the preparation of syngrafts (isografts), allografts and/or xenografts. These methods might include in vitro differentiation of embryonic cells for therapeutic transplantation into a patient, including situations where the cells are genetically modified to correct a medical defect. Such applications might include treatment of diseases such as diabetes, Parkinson's disease, motor neurone disease, multiple sclerosis, AIDS etc, or disease conditions characterised by a loss of function in the cells or an organ of an affected individual.

The present invention therefore also extends to the provision of a kit for the diagnosis of Large Offspring Syndrome (LOS) in an animal embryo or in a sample from the embryo comprising a means for the detection of Igf2r gene expression. Such kits may also be used to carry out a method in accordance with the ninth aspect of the present invention of a method of testing an embryo culture environment for the induction of Large Offspring Syndrome in an animal embryo. Analysis or detection of Igf2r expression may be carried out as described above.

Preferred features for the second and subsequent aspects of the invention are as for the first aspect mutatis mutandis.

The invention will now be further described by way of reference to the following Examples and Figures which are provided for the purposes of illustration only and are not to be construed as being limiting on the invention. Reference is made to a number of Figures in which:

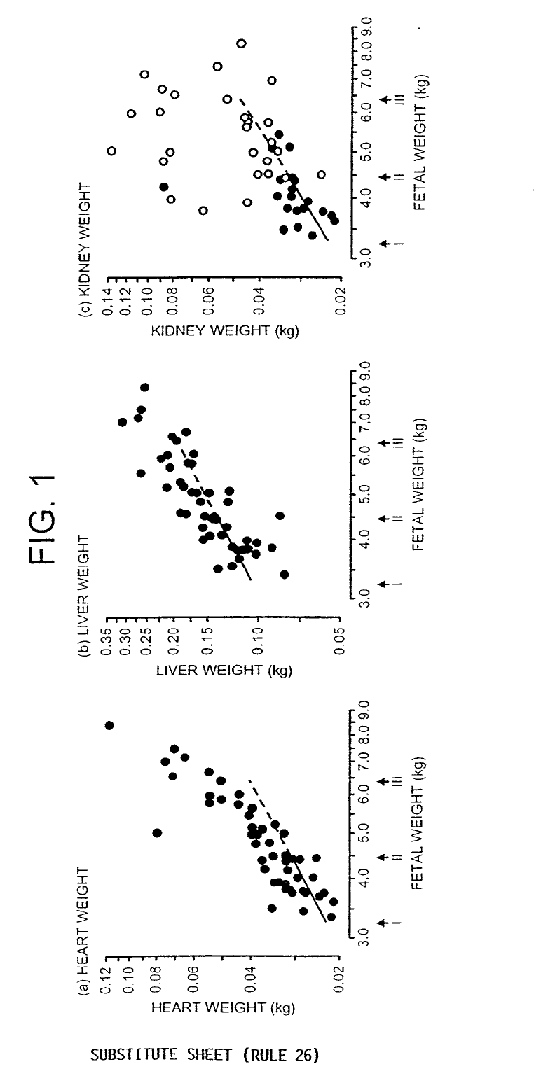

FIG. 1 shows differential organ growth of the fetal liver (FIG. 1(a)), heart (FIG. 1(b)), kidneys (FIG. 1(c)) at day 125 of gestation following in vitro culture. Control (CONT) fetuses are represented by the solid (fit to data) and dotted (predicted growth to term) lines, generated by the linear allometric equation logₑy=logₑa+b(logₑ)x, where y=organ weight and x=fetal weight. Points (i) and (ii) represent, respectively, the minimum and maximum weights of CONT fetuses at day 125 of gestation, and point (iii) represents their birth weights predicted from the Gompertz equation. Allometric coefficients (b±s.e.m.) are for heart (CONT b=0.91±0.18, Culture b=1.47±0.12), liver (CONT b=0.89±0.13, Culture b=1.16±0.10) and kidneys (CONT b=1.10±0.29, Culture b=1.09±0.26), (●, ◯). Open data points (◯) represent pregnancies where polyhydramnios was present.

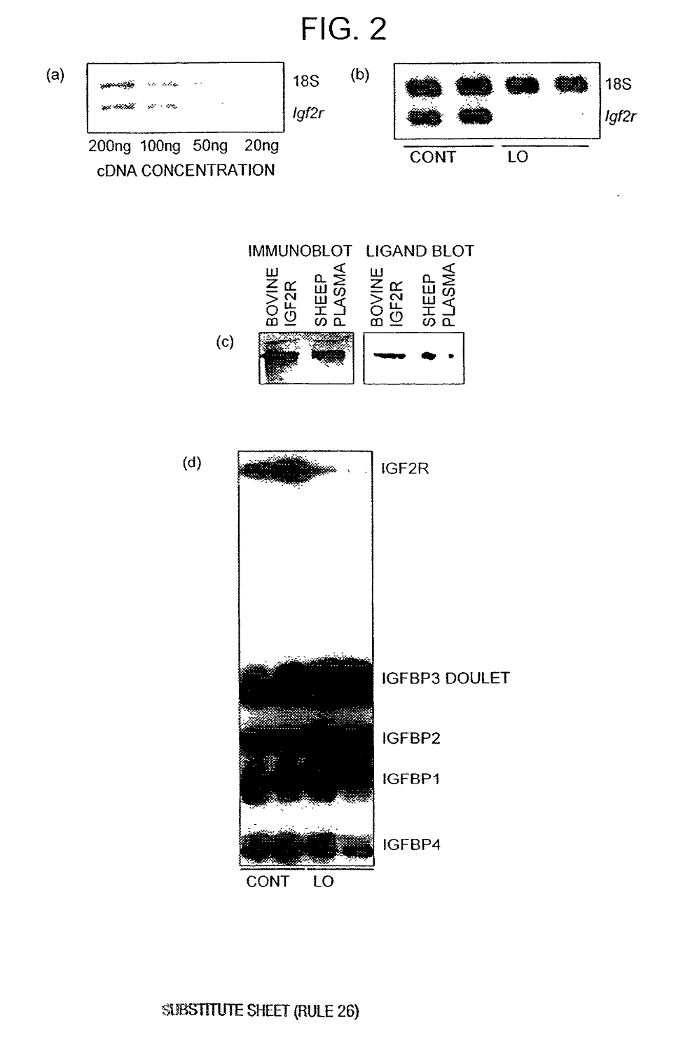

FIG. 2 shows the results of the validation of Igf2r PCR assay and identity of IGFIIr protein.

FIG. 2(a) shows a representative validation of reverse-transcription polymerase chain reaction (RT-PCR) assay to quantify levels of gene transcription (in this case liver Igf2r) relative to the 18S ribosomal subunit. Both Igf2r and 18S were amplified in a single tube, with an appropriate cycle number and ratio of 18S primer to attenuating Competimer™ to ensure linear amplification over at least a 10-fold dilution of cDNA. Values are arbitrary units of signal intensity.

FIG. 2(b) shows RT-PCR amplifications representing Igf2r and 18S in CONT and LO liver samples.

FIG. 2(c) shows confirmation of a >200 kDa band on IGFII Western ligand blot as IGFII receptor. Immunoblotting of sheep fetal plasma using a polyclonal antibody against IGFIIr protein reveals a single band (a) which co-migrates with purified bovine IGFIIr (b) and the >200 kDa band identified by Western ligand blot of fetal plasma.

FIG. 2(d) shows IGFII Western ligand blots representing CONT and LO plasma samples, identifying IGFIIr protein and the IGF binding proteins (IGFBP).

### MATERIALS AND METHODS

Model System for LOS in Sheep

A variety of in vitro embryo culture systems have been associated with programming of the syndrome (Lau et al, Genes Dev. 8 2953-2963 (1994)) although the incidence has been neither predictable nor reproducible. In the present studies fertilised eggs from superovulated Scottish Blackface ewes were cultured for five days in four in vitro treatments. These comprised two cultured systems, synthetic oviduct fluid (SOF) and TCM 199 co-culture with bovine granulosa cell monolayers (Cocult) (Lau et al, Genes Dev. 8 2953-2963 (1994)), supplemented with one of two sera (A and B) obtained from steers on different dietary regimes (treatments SOFA, SOFB, CocultA and CocultB). Blastocyst stage embryos were transferred singly to Scottish Blackface recipient ewes and conceptuses were recovered at day 125 of gestation (11 SOFA, 13 SOFB, 11 CocultA and 13 CocultB). Day 6 control (CONT) embryos were transferred to recipients 1-6 hours after recovery, with 22 conceptuses recovered

### Embryo Culture and Transfer

Superovulation was induced in mature Scottish Blackface ewes using 400 i.u. PMSG (Intervet) and Ovagen (ICP) after oestrous synchronisation with Chronogest intravaginal sponges (Intervet). Estrous cycles were synchronised in both donor and recipient ewes using progesterone-releasing intravaginal sponges (30 mg fluorogestone acetate, Chronogest, Intervet) for 12 days. For recipient ewes the same sponge remained in position for 12 days whereas for donor ewes it was removed and a second sponge inserted after 7 days. Recipients received 400 i.u. PMSG (Intervet) at sponge withdrawal. Estrous observations began 24 hours after progesterone withdrawal and were carried out at 08.00, 12.00, 16.00 and 24.00 h during the next three days, using raddled vasectomised rams. Superovulation was induced in donor ewes using a total dose of 9 mg of oFSH (Ovagen, ICP Ltd, New Zealand) administered twice daily in equal doses over four days beginning on day 10 of the 12 day progesterone priming period.

Laparoscopic intrauterine insemination was carried out 44 hours after sponge withdrawal using fresh semen from a single Suffolk sire to minimise genetic variation. Semen was diluted 3:1 in phosphate-buffered saline containing 1000 i.u. sodium penicillin and 1 mg streptomycin sulphate per ml. The diluted semen was held at 30° C. prior to insemination and approximately 100×10⁶ motile spermatozoa were placed in each uterine horn.

Embryos were recovered by laparotomy 36 hours after insemination following induction of general anaesthesia (Halothane: Oxygen; Nitrous oxide). Each oviduct was flushed with 20 ml HEPES-buffered synthetic oviductal fluid (HSOF), derived from the formulation of Tervit et al (1972), and containing 20.0 mM HEPES buffer, 5.0 mM sodium bicarbonate, 1.5 mM glucose, 3.3 mM sodium lactate, 0.33 mM sodium pyruvate, 1.0 mM L-glutamine and 30 mg/mi bovine serum albumin.

Embryos were then allocated to the four culture treatments. Allocation across treatments aimed to ensure that, in so far as egg yields permitted, each donor was equally represented. Embryo culture was in SOF and granulosa cell co-culture (Cocult) according to standard techniques. The co-culture medium used was Medium 199 with Earle's salts (Life Technologies, UK) together with 50 i.u./ml penicillin, 50 μg/ml streptomycin sulphate and 10% heat-inactivated (56° C.; 30 min) steer serum (Globepharm Ltd., UK). The pH was adjusted to 7.3 and osmolarity to between 390 and 310 mOsm. SOF was derived from the original formulations of Tervit et al (1972) excluding HEPES buffer and including 25.0 mM sodium bicarbonate, 9.9 mM sodium lactate, 0.99 mM sodium pyruvate, 1.5 mM glucose, 1.0 mM L-glutamine, 50 i.u./ml penicillin and 50 μg/ml streptomycin sulphate. The pH of SOF medium was adjusted to 7.4 and osmolarity to between 270 and 280 mOsm or 290 and 310 mOsm.

Sera A and B were obtained from steers fed on diets including 120 and 190 g crude protein/kg dry matter, respectively. These sera were used (4:1 v/v) to supplement both SOF and Cocult, forming four embryo culture treatments (SOFA, SOFB, CocultA and CocultB). Each 60 mm diameter tissue culture dish (Bibby Sterilin) used for co-culture contained 50 μl droplets (n=8) of M199 and serum overlaid with mineral oil (Cat. No. M8410 Sigma, UK). At the time of zygote introduction (Day 1), 4-day old bovine granulosa cell layers were established in these droplets. The culture medium was refreshed (50% replacement, by volume) just before the zygotes were introduced and thereafter at 48-hour intervals during the culture period, which proceeded for 5 days in a humidified atmosphere (5% CO₂, 95% air) at 38.5° C. Serum-supplemented SOF droplets (20 μl under oil) were prepared on the day of zygote collection, with 8 droplets used per 60 mm culture dish. Up to 4 zygotes were cultured per drop in a reduced-oxygen atmosphere (5% CO₂, 5% O₂, 90% N₂) at 38.5° C. At 48-hour intervals thereafter, rather than refreshing the media in each droplet, zygotes were moved to freshly prepared 20 μl droplets under identical conditions.

After five days in embryo culture, embryos which had developed to late morula/blastocyst stage were transferred singly to uteri of oestrus synchronised ewes by laparoscopy. The transfer technique involved temporary exposure of the tip of the uterine horn through a small ventral incision of the abdominal wall by laparoscopically-guided pick-up. Pregnant ewes were euthanased at day 125 of gestation by administration of 25 ml of a 20% w/v solution of pentobarbitone sodium (Euthatal, Rhone Merieux Ltd, UK). The gravid uterus was then recovered and weighed. The fetus was removed and weighed and the liver, heart, kidneys and individual muscles were excised and weighed.

All ewes in this study were maintained on hay treated with 2.5% urea. Procedures were carried out in strict accordance with Home Office regulations. Fetal weights were analysed by ANOVA. Differences between means and allometric coefficients were then assessed by t-tests.

### Results

Fetal weights in all embryo culture treatments were greater than in the control group at day 125 of gestation (P<0.01; mean±s.e. CONT 3907±68, SOFA 4594±248, SOFB 4856±309, CocultA 4495±291 and Cocult 5302±422) and the percentages of fetuses heavier than the heaviest control group fetus were 46, 54, 36 and 54% respectively. Polyhydramnios (defined as fetal fluid weights more than three standard deviations above the mean for control group conceptuses) was evident in 54% of pregnancies derived from in vitro cultured embryos and 15% displayed severe fetal edema (Hydrops fetalis). Three fetuses from culture treatments were twice the mean weight of the control group, indicating extreme overgrowth. Furthermore, 13 % of fetuses from cultured embryos were heavier at day 125 of gestation then the predicted birthweight of the control fetuses three weeks later at day 147. The external appearance of many large fetuses from the culture treatments was similar to that of a term fetus, both in general conformation and extent of coat growth. This suggests that development is considerably advanced by LOS.

The development of key organs in the oversized fetuses was also found to be fundamentally perturbed. Development at day 125 was assessed by relating organ weight to fetal body weight for all treatment groups using the linear allometric equation:

logₑy=logₑa+blogₑx

where y=organ weight and x=fetal weight.

Allometric coefficients (b±s.e.m.) established for the control (CONT) group shown in FIG. 1 were similar to those derived from 121 normal fetuses of the same genotype recovered between days 35 and 135 of gestation in an earlier study (Sinclair et al Theriogenology 45 223 (1996)) (heart b=0.92±0.01, liver b=0.86±0.01, kidneys b=0.99±0.01). Corresponding coefficients for fetuses derived from all in vitro cultured embryos (Culture: FIG. 1) were compared to those from the previous study since the larger weight range of fetuses observed therein results in more precise estimates of allometric coefficients. These growth coefficients were significantly greater for liver (FIG. 1(a), P<0.05) and heart (FIG. 1(b), P<0.0001) from fetuses derived from in vitro cultured embryos, indicating that these organs were growing at a higher rate than expected from both normal fetuses at day 125 of gestation and from older, normally-developing, fetuses of equivalent weights. Kidney weights from cultured embryos were highly variable (FIG. 1(c)). Several kidneys were grossly enlarged and these were generally associated with polyhydramnios (defined as fetal fluid weights more than three standard deviations above the mean for CONT pregnancies; FIG. 1(c)) and severe fetal oedema (Hydrops fetalis; data not shown). Such alterations in development of vital organs may contribute to the increased prenatal and perinatal losses associated with the Large Offspring Syndrome.

### EXAMPLE 1

### Analysis of Igf2r Gene Expression

Transcript levels were quantified relative to 18S ribosomal RNA in a single tube assay. Amplification by 18S primers was attenuated by competition with 18S PCR Competimer™'s (Ambion) which block extension by Taq DNA polymerise. Total RNA was extracted from tissues using RNA Isolator (TM) (Genosys) and 2.5 μg reverse transcribed using pd(N)6 random hexamer primer and First-strand cDNA synthesis kit (Pharmacia Biotech). In a 25 μl PCR reaction, 125 ng of cDNA template was mixed with 0.5 μM gene-specific primers, 0.5 μM alternative 18S PCR Primers and 18S PCR Competimer™'s (Ambion), 10×buffer with 2.5 mM MgCl₂, 1 mM dNTPs (InVitrogen) and 0.635 units Taq DNA Polymerase (Boehringer). Primers for Igf2 exon-specific transcripts (Ohlsen et al DNA Cell Biol. 13 (4) 377-388 (1994)), IGFBP2, IGFBP3 (Winger et al Biol. Reprod. 56 1415-1423 (1997)) and IGFBP4 (Armstrong et al Endocrinology 139 (4) 2146-2154 (1998)) were as described previously while Igf2r primers were designed from Genbank bovine sequence J03527 (5′-nucleotides 530-551, 3′-nucleotides 769-750; Lobel et al J. Biol. Chem. 263 (5) 2563-2570 (1988)). To ensure that the assay was in the linear range of amplification the ratio of 18S Competimer™: 18S Primer was varied for each tissue and gene-specific primer set. All of the Igf2 primers span introns, providing a control for contaminating DNA and tubes with no cDNA were also included in each PCR reaction.

In the protocol used, cDNA was denatured for 5 mins at 95° C., then subjected to PCR (94° C. for 1 min, 60-65° C. for 30s, 72° C. for 1 min) for the minimum number of cycles required to detect transcripts on a 1.7% agarose gel stained with ethidium bromide (typically 25-27 cycles). Band intensities were quantified using a molecular Imager image analysis system with Molecular Anal software (Bio-Rad). Results are exposed as ratios of transcript: 18S and statistical differences determined using TWO-SAMPLE t-test (Minitab). PCR product identify was confirmed by sequencing (Sequence Version 2.0). Amersham).

### Results

Fetuses exceeding 5.5 kg in all treatment groups were defined as large offspring (LO) for analysis of gene expression. Using relative RT-PCR (FIG. 2(a)), the levels of expression of Igf2 and Igf2r in the liver, kidney and heart were compared from the CONT and LO offspring fetuses. Tissue from the forelimb extensor carpi radialis forelimb was also used for the analysis of gene expression in a skeletal muscle. Igf2 is expressed as a series of alternatively-spliced, exon-specific transcripts in a tissue-specific and developmental stage-specific manner (Ohlsen et al DNA Cell Biol. 13 (4) 377-388 (1994)). Expression of all possible transcripts was examined using 5′-primers representing each non-coding exon and a 3′-primer from the first coding exon, exon 8. No quantitative changes were detected in the level of any exon-specific transcript normally expressed in the day 125 sheep fetus (transcripts from leader exons 5, 6 and 7) shown in Table 1. Furthermore, no qualitative changes were observed which would have indicated activation of exon-specific transcript forms not normally transcribed in the tissues examined at day 125 of gestation (i.e. exons 1 and 3-postnatal transcripts and exon 4-placental transcripts; data not shown). Thus the mechanism underlying ovine Large Offspring syndrome does not appear analogous to the fetal overgrowth typical of Beckwith-Weidmann syndrome in humans, induced by overexpression of Igf2. In contrast, expression of the Igf2r gene was significantly reduced by ˜50% in the liver and muscle and by ˜30% in the kidney and heart (Table 1).

Table 1 shows the expression of gene transcripts in day 125 fetal tissues. Values are arbitrary absorbance units expressed as a ratio of transcript: 18S. All values are mean±se for at least 21 CONT and 10 LO fetuses. The asterisks *,**,*** indicate values for which there is a significant difference between the CONT and LO values (Students t-test P<0.05, 0.01, 0.001 respectively). The abbreviation BQ indicates that amplification of gene transcripts were below detection limit of assay (18S amplification could not be modulated sufficiently by competitors to ensure linear amplification of both transcript and 18S).

### EXAMPLE 2

### Western Blot Analysis

To investigate whether decreased Igf2r gene expression resulted in reduced IGFIIr protein levels, Western ligand blots of tissue protein extracts and plasma samples were probed with ¹²⁵I-IGFII. Western ligand blotting using ¹²⁵I-IGFII was performed as described previously (Armstrong et al J. Endocrinol. 120 373-378 (1989)), with the modification that the cathode buffer used for transfer to nitrocellulose contained 0.5 mg/ml BSA (Bhavsar et al Anal. Biochem. 221 234-242 (1994)). IGFBP's were identified as previously described (Delhanty, P. J. D. and Van, V. K. M., Endocrinology 132 (1) 41-52 (1993)). Immunoblotting was performed with an anti-bovine IGFII receptor antibody using a peroxidase detection system (Vector Labs, UK). The IGFIIr antibody and purified IGFIIr were provided by Dr P Lobel, Centre for Advanced Biotechnology and Medicine, Piscataway, N.J., USA.

Previous authors have speculated that a ˜250 kDa protein evident on ligand blots was the IGFIIr (Butler, J. H. and Gluckman, P. D., J. Endocrinol. 109 333-338 (1986); Delhanty, P. J. D. and Han, V. K. M., Endocrinol. 145 545-557 (1993)) and this was confirmed using Western blotting with an anti-bovine IGFIIr antibody

(FIG. 2(b)). Reductions of 61% and 81% in muscle and liver IGFIIr protein levels were observed, respectively (P<0.001, Table 2) and 67% reduction in the circulating form (P<0.001, Table 2). In view of the reduction in Igf2r expression and resulting overgrowth phenotype in mice with disrupted Igf2r expression, these findings provide strong evidence for a direct causative role for IGF2R in LOS in sheep. The dramatic decrease in both circulatory and tissue IGFIIr levels could account for the widespread organ effects as well as the general overgrowth.

Table 2 shows the levels of IGFII receptor protein, IGFBP2 and IGFBP3 in day 125 fetal plasma and tissues. All values are mean±s.e×1000 from at least 21 Control and 10 LO fetuses. Values are phosphorimager absorbance units of signal intensity from IGFII Western ligand blots. The asterisks (*,**,***) indicate values for which there is a significant difference between the CONT and LO values (Students t-test P<0.05, 0.01, 0.001 respectively).

### EXAMPLE 3

### Assay of Plasma Levels of IGF-II

The mechanisms by which IGFIIr can influence fetal growth have not been fully established. One function of IGFIIr is to target IGFII to the lysosymes for degradation. Thus it is widely assumed from mouse studies that a reduction in IGFIIr impairs clearance of IGFII from the circulation. The resulting elevation in levels of this potent fetal mitogen provides a well-established mechanism for increasing fetal growth through cell proliferation and inhibition of apoptosis. Plasma IGFII was measured after acid HPLC (Gutierrez et al J. Endocrinol. 153 231-240 (1997)) using an anti-human polyclonal IGFII antiserum and human recombinant IGFII as standard (GroPep Pty Ltd). The detection limit of the assay was approximately 30 pg/tube.

### Results

However, it was surprisingly found in the present studies that plasma levels of IGFII were not significantly different between the CONT and LO sheep fetuses (6.08±0.57 and 4.63±0.67 ug/ml respectively; p=0.1). This result may be attributable to a species-specific difference in response to depressed IGFIIr, since Igf2r null mutant mice at an equivalent stage of gestation (E18.5) were reported to have a four-fold higher level of serum IGFII than wild-types (Ludwig et al Dev. Biol. 177 517-535 (1996)). However, this difference could have been introduced by inefficient removal of the increased circulating IGF binding proteins observed in the mutant mice. In addition, none of the differences in serum IGFII reported in this and other mouse studies were statistically significant (Eggenschwiler et al Genes Dev. 11 3128-3142 (1997); Lau et al Genes Dev. 8 2953-2936 (1994)). Furthermore in H19 null mutant mice, where elevated expression of Igf2 is responsible for the fetal overgrowth, serum IGFII is not elevated, although tissue levels are increased (Eggenschwiler et al Genes Dev. 11 3128-3142 (1997)).

### Discussion

Absolute changes in IGFII levels are not essential for altered biological activity of this growth factor. Action of the insulin-like growth factors is complex, mediated by a series of binding proteins (IGFBP's) and proteases (Jones, J. I. and Clemmons, D. R. Endocrine Reviews 16 3-34 (1995)). The Western ligand blots in the present study revealed that circulating levels of IGFBP2, which binds most avidly to IGFII (Delhanty, P. J. D. and Han, V. K. M. Endocrinology 132 (1) 41-52 (1993)), were increased by 102% in the LO fetuses (Table 2) and this was accompanied by an 86% increase in liver gene expression (Table 1). IGFBP2 has been suggested to prolong IGFII half-life in the circulation and to transport IGFII between blood and tissue fluid, modifying local concentrations and access to receptors. Thus increased IGFBP2 in LO fetuses may increase bioactivity of IGFII without a change in ligand concentration. Plasma levels of IGFBP3, previously reported to be increased in the plasma of oversized E18/19 fetal mice (Lau et al Genes Dev. 8 2953-2963 (1994)), were not changed in the large ovine fetuses and expression levels remained unchanged in all tissues examined except the kidney, where there was a 23% reduction in transcript levels. No differences in IGFBP4 expression was detected in any of the tissues examined.

It is also possible that reduced Igf2r expression in large fetuses increases fetal growth by a mechanism independent of IGFII. In addition to effects on clearance of IGFII, IGFIIr is essential for activation of TGFB1, which exerts potent growth suppressive effects (Wang et al Cancer Res. 57 2543-2546 (1997)), and may account for some or all of the overgrowth observed in LOS. The IGFIIr also has a mannose 6-phosphate binding domain and delivers approximately fifty mannose 6-phosphate tagged lysosomal enzymes to lysosomes (Sklar et al J. Biol. Chem. 264 16733-16738 (1989)). Reduced levels of several lysosomal enzymes was reported in cells from Igf2r null mice (Wang et al Nature 372 464-467 (1994)), with likely consequences for tissue remodelling during fetal development (Sklar et al (1989)). Thus any disruption to lysosomal enzyme function caused by reduced IGFIlr in LOS may explain some of the observed organ defects, and may also contribute to the gross overgrowth. Finally, of course, a combination of effects of the multifunctional IGFII/mannose-6 phosphate receptor may be responsible for the complex phenotype observed in LOS in sheep.

In this study, a model for large offspring syndrome in sheep has been established, which is likely to be applicable to a similar syndrome in cattle (Kruip, T. A. M. and den Daas, J. H. G. Theriogenology 47 43-52 (1996)). The high incidence of large offspring observed from the embryo culture treatments in the present studies has allowed a comprehensive phenotypic description and the first report of genes implicated in the perturbing mechanism. Identification of downregulated Igf2r expression makes this imprinted gene a strong candidate for initiating the syndrome due to epigenetic change induced by culture conditions in the pre-implantation embryo and this is currently under investigation. Further understanding of the mechanism should lead to the development of embryo manipulation protocols which avoid the problem. This is of critical importance to the development of both cloning technologies and in vitro embryo production in domestic species. Furthermore, since it is not known why similar perturbations have not been described after embryo manipulations in non-ruminants, large offspring syndrome has implications for new assisted reproduction protocols in humans.

TABLE 1
Gene Transcript Levels in day 125 fetal tissues

| Organ | Embryo | Igf2 exon5 | Igf2 exon6 | Igf2 exon7 | Igf2r | IgfBP2 | IgfBP3 | IgfBP4 |
|---|---|---|---|---|---|---|---|---|
| Liver | Control | 1.79 ± 0.11 | 0.77 ± 0.05 | 1.03 ± 0.07 | 0.63 ± 0.09 | 0.40 ± 0.08 | 0.48 ± 0.05 | 0.45 ± 0.09 |
| | LO | 1.57 ± 0.25 | 1.00 ± 0.12 | 1.05 ± 0.04 | 0.26 ± 0.06 | 0.75 ± 0.13 | 0.53 ± 0.06 | 0.31 ± 0.07 |
| | | | | | *** | * | | |
| Kidney | Control | BQ | 0.60 ± 0.03 | 1.55 ± 0.12 | 0.46 ± 0.03 | 0.51 ± 0.05 | 1.01 ± 0.06 | 0.56 ± 0.03 |
| | LO | BQ | 0.57 ± 0.03 | 1.41 ± 0.18 | 0.31 ± 0.04 | 0.38 ± 0.05 | 0.78 ± 0.07 | 0.61 ± 0.04 |
| | | | | | * | | * | |
| Heart | Control | BQ | BQ | 1.89 ± 0.17 | 0.82 ± 0.03 | BQ | 0.78 ± 0.05 | 0.35 ± 0.05 |
| | LO | BQ | BQ | 1.98 ± 0.24 | 0.57 ± 0.05 | BQ | 0.84 ± 0.08 | 0.32 ± 0.06 |
| | | | | | *** | | | |
| Muscle | Control | 1.86 ± 0.12 | 1.14 ± 0.06 | 0.63 ± 0.05 | 0.41 ± 0.03 | BQ | 0.46 ± 0.06 | 0.82 ± 0.07 |
| | LO | 2.30 ± 0.23 | 1.19 ± 0.07 | 0.63 ± 0.07 | 0.21 ± 0.03 | BQ | 0.56 ± 0.15 | 0.96 ± 0.11 |
| | | | | | *** | | | |

TABLE 2
IGF-IIR and IGF Binding Proteins in day 125 fetal tissues

| Organ | Embryo | IGF-IIR | IGFBP2 | IGFBP3 |
|---|---|---|---|---|
| Plasma | Control | 116.9 ± 15.7 | 127.3 ± 77.0 | 198.8 ± 23.1 |
| | LO | 385.3 ± 10.6 | 256.6 ± 167.2 | 281.3 ± 58.1 |
| | | *** | ** | |
| Liver | Control | 1297.5 ± 152.0 | 158.0 ± 19.0 | 427.7 ± 47.7 |
| | LO | 242.8 ± 42.2 | 188.1 ± 31.6 | 329.4 ± 35.3 |
| | | *** | | |
| Kidney | Control | 2537.3 ± 608.7 | 434.8 ± 84.5 | 333.3 ± 68.5 |
| | LO | 2026.1 ± 889.0 | 471.7 ± 130.2 | 262.4 ± 61.8 |
| Heart | Control | 1220.6 ± 120.1 | 307.3 ± 39.3 | 379.9 ± 43.6 |
| | LO | 836.4 ± 170.0 | 263.9 ± 49.7 | 308.6 ± 33.3 |
| Muscle | Control | 174.7 ± 12.6 | 94.8 ± 10.0 | 110.4 ± 11.2 |
| | LO | 68.3 ± 9.4 | 91.3 ± 12.5 | 105.1 ± 12.7 |
| | | *** | | |

1. A method of screening an animal embryo for Large Offspring Syndrome (LOS) comprising the step of analysing Insulin-like growth factor-2 receptor (Igf2r) gene expression in the animal embryo or in a biological sample from the embryo.

2. A method as claimed in claim 1, in which the animal is a mammal.

3. A method as claimed in claim 2, in which the mammal is a human.

4. A method as claimed in claim 2, in which the mammal is of an ungulate species.

5. A method as claimed in claim 4, in which the ungulate species is cattle, sheep, pigs, goats, horses, camels or buffalo.

6. A method as claimed in claim 4 or claim 5, in which the ungulate mammal is a transgenic animal.

7. A method as claimed in any one of claims 1 to 6, in which the biological sample comprises a tissue biopsy, cells or cellular material or a biological fluid.

8. A method for in vitro production of an animal embryo comprising the steps of introducing a sperm cell to the oocyte in an in vitro culture system, subsequently culturing the resulting embryo and screening the embryo for Large Offspring Syndrome (LOS) by analysing Igf2r gene expression in the animal embryo or in a biological sample from the embryo.

9. A method as claimed in claim 8, in which the method includes the additional step of maturing the oocyte in vitro prior to fertilisation.

10. A method for reconstituting an animal embryo comprising the step of transferring a nucleus from a donor cell into a suitable recipient cell and screening the resulting embryo for Large Offspring Syndrome (LOS) by analysing Igf2r gene expression in the animal embryo or in a biological sample from the embryo.

11. A method of reconstituting an animal embryo, the process comprising transferring a diploid nucleus into an oocyte which is arrested in the metaphase of the second meiotic division without concomitantly activating the oocyte, keeping the nucleus exposed to the cytoplasm of the recipient for a period of time sufficient for the embryo to become capable of giving rise to a live birth and subsequently activating the reconstituted embryo while maintaining correct ploidy, including screening the embryo for Large Offspring Syndrome (LOS) by analysing Igf2r gene expression in the animal embryo or in a biological sample from the embryo.

12. A reconstituted animal embryo prepared by a method as defined in claim 10 or claim 11.

13. A method for preparing a non-human animal, the method comprising:
(a) culturing an animal embryo in vitro where the embryo has been prepared by in vitro production or by reconstituting an animal embryo as defined in any one of claims 8 to 11;
(b) screening the embryo for Large Offspring Syndrome (LOS) by analysing Igf2r gene expression in the embryo or in a biological sample from the embryo;
(c) causing an animal to develop to term from the embryo; and
(d) optionally breeding from the animal so formed.

14. A non-human animal prepared as defined in claim 13.

15. A method of screening an animal cell for Large Offspring Syndrome comprising the step of analysing Igf2r gene expression in the animal cell.

16. A method of testing an embryo culture environment for the induction of Large Offspring Syndrome in an animal embryo comprising the step of analysing Igf2r gene expression in the animal embryo or in a biological sample from the embryo.

17. A method of therapy comprising the administration of animal cells to a patient wherein the cells have been prepared from an embryo screened by a method as defined in any one of claims 1 to 7.

18. The use of a nucleic acid sequence encoding Insulin-like growth factor-2 receptor (IGF2R) or a fragment thereof or a sequence complementary or homologous thereto in the preparation of an agent for the diagnosis of Large Offspring Syndrome (LOS) in an animal embryo or in a sample from the embryo.

19. The use of cell or a cell line derived from an embryo screened by a method as defined in any one of claims 1 to 7 in medicine.

20. The use of a cell or a cell line derived from an embryo screened by a method as defined in any one of claims 1 to 7 in the preparation of a cell graft for transplantation.

21. A kit for the diagnosis of Large Offspring Syndrome (LOS) in an animal embryo or in a sample from the embryo comprising a means for the detection of Igf2r gene expression.
